(19) United States
(12) Patent Application Publication
(10) Pub. No.: US 2009/0238767 A1
(43) Pub. Date: Sep. 24, 2009

(54) TARGETING CONTRAST AGENTS OR TARGETING THERAPEUTIC AGENTS FOR MOLECULAR IMAGING AND THERAPY

(75) Inventors: Helga Hummel, Aachen (DE); Volker Ulrich Weiler, Aachen (DE); Ralf Hoffmann, Leverkusen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/721,382

(22) PCT Filed: Dec. 12, 2005
(86) PCT No.: PCT/IB05/54185

§ 371 Date: Jun. 11, 2007

(30) Foreign Application Priority Data
Dec. 17, 2004 (EP) 04106694.5

(51) Int. Cl. A61K 49/00; B05D 3/00; B32B 5/16

(52) U.S. Cl. 424/9.32; 427/2.13; 428/402.2

(57) ABSTRACT

This invention discloses a method of synthesizing targeting contrast agents for molecular imaging and targeting diagnosis and therapy, targeting contrast agents and targeting therapeutic agents and their use.

The present invention relates to targeting contrast agents and targeting therapeutic agents, and to methods for their production and use.

Known imaging techniques with a tremendous importance in medical diagnostics are positron emission tomography (PET), computed tomography (CT), magnetic resonance imaging (MRI), single photon computed tomography (SPECT) and ultrasound (US). Although today's imaging technologies are well developed, they rely mostly on non-specific, macroscopic, physical, physiological, or metabolic changes that differentiate pathological from normal tissue.

Targeting molecular imaging (MI) has the potential to reach a new dimension in medical diagnostics. The term “targeting” is related to the selective and high specific binding of a natural or synthetic ligand (binder) to a molecule of interest (molecular target) in vitro or in vivo.

MI is a rapidly emerging biomedical research discipline that may be defined as the visual representation, characterization and quantification of biological processes at the cellular and sub-cellular levels within intact living organisms. It is a novel multidisciplinary field, in which the produced images reflect cellular and molecular pathways and in vivo mechanisms of disease present within the context of physiologically authentic environments rather than that they identify molecular events responsible for disease.

Several different contrast-enhancing agents are known today and their non-specific or non-targeting forms are already in clinical routine. Some examples mentioned below are reported in literature.

For example, Gd-complexes could be used as contrast agents for MRI according to “Contrast Agents I” by W. Krause (Springer Verlag 2002, page 1 and following pages). Furthermore, superparamagnetic particles are another example of contrast-enhancing agents, which could also be used as contrast agents for MRI (Textbook of Contrast Media, Superparamagnetic Oxides, Dawson, Cosgrove and Grainger Isis Medical Media Ltd, 1999, page 373 and following pages). As described in “Contrast Agents II” by W. Krause (Springer Verlag 2002, page 73 and following pages), gas-filled micro bubbles could be used in a similar way as contrast agents for ultrasound. Moreover, “Contrast Agents II” by W. Krause (Springer Verlag, 2002, page 151 and following pages) reports the use of iodinated liposomes or fatty acids as contrast agents for X-Ray imaging.

Contrast-enhancing agents that can be used in functional imaging are mainly developed for PET and SPECT.

One example of these contrast agents is ¹⁸F-labelled molecules such as desoxyglucose (Beuthien-Baumann B, et al., (2000), Carbohydr. Res., 327, 107). The use of these labeled molecules as contrast agents for PET is described in “Contrast Agents II” by W. Krause (Springer Verlag, 2002, page 201 and following pages). However, they only accumulate in tumor tissue without any prior specific cell interaction. Furthermore, ⁹⁹Tc-labeled molecules such as antibodies or peptides could be used as targeting contrast agents for SPECT (Verbruggen A. M., Nosco D. L., Van Nerom C. G. et al., ^{99m}Tc-L,L-ethylene dicysteine: a renal imaging agent, Nucl. Med. 1992, 33, 551-557), but the labeling of such complex molecules is very difficult and cost-intensive.

The same can be said for several other ligands already existing for use in PET/SPECT, e.g. L-DOPA (dopamine receptor, Parkinson) (Luxen A., Guillaume M, Melega W P, Pike V W, Solin O, Wagner R, (1992) Int. J. Rad. Appl. Instrum. B 19, 149); Serotonin analogue (serotonin receptor) (Dyck C H, et al., 2000, J. Nucl. Med., 41, 234); Somatostatin analogue (somatostatin, oncology) (Maecke, H. R. et al., Eur. J. Nucl. Med. Mol. Imaging, 2004, Mar. 17), Peptide for integrin receptors (angiogenesis) (Wicklinde, S. A. et al., Cancer Res., 2003 Sep. 15, 63(18), 5838-43; Wicklinde, S. A. et al., Circulation 2003 Nov. 4, 108, (18), 2270-4).

Moreover, the Cu-catalyzed reaction of imidazoles with aryl boronic acids (e.g. Collman, Zhong, Organic Letters, 2000, vol. 2, no. 9, 1233-1236) has also been reported.

However, the identification of specific molecular events responsible for disease is becoming increasingly important in medicine. Targeting agents which are equipped with molecular recognition mechanisms to enrich contrast-enhancing materials specifically in certain tissues in vivo or in vitro and allowing insight into molecular pathology are therefore essential in diagnosis and future therapy as well.

Thus, it is an object of the present invention to provide a new generation of improved contrast agents which allow an early diagnosis with high sensitivity and specificity as well as a differential diagnosis, and to provide less costly and time-consuming methods for producing said improved contrast agents. In this respect, it would also be advantageous to provide production processes and targeting agents produced thereby which can be easily adapted to actually occurring problems which have to be solved within a short time and with low effort concerning cost and man power. Apart from their potential for imaging diagnostics, targeting contrast agents will also play a crucial role in the development of new therapeutics. Such targeting contrast agents are currently not available.

The object of the present invention is advantageously solved by the present invention as described below and additionally defined in the claims and examples. Preferred, non-limiting variants are described in the Figures and used for explanation of the invention.

The present invention relates to a method for the production of a targeting contrast agent or a therapeutic agent, the method comprising the steps of

a) providing a core;
b) optionally adding a shell to the core;
c) modifying the core or the shell by attaching at least one molecule of a binding unit; and
d) linking at least one ligand, bearing at least one imidazole functionality, to the modified core or the modified shell by using an appropriate catalyst.

In a further embodiment of said method, more than one shell can be added to the core in step b). In other words, the outer shell can be separated from the core by one to several inner shells. In preferred embodiments of the present invention, the core can be separated from the outer shell by 1 to 100 inner shells, more preferably by 1 to 50 inner shells. The shell or shells may comprise a monolayer or a polylayer. Each of these shells (which may comprise a monolayer or a polylayer of an appropriate material in preferred embodiments of the present invention) has a thickness of about 0.5 nm to 100 nm. In a preferred embodiment of the present invention, each shell has a thickness of about 0.5 nm to 500 nm. Furthermore, each shell or even several shells may comprise the same material or different materials.

In a further variant of the present invention, the shell or shells may cover the core at least partially. This is preferably the case when e.g. an organic polymer (e.g. polyethylene glycol/PEG, polyvinyl alcohol/PVA, polyamide, polyacrylate, polyurea), an organic polymer with functional end groups (e.g. 1,2-distearoyl-sn-glycero-3-phosphoethanolamine-N-[carboxy(polyethylene glycol)2000]ammonium salt), a biopolymer (e.g. polysaccharide such as dextran, xylan, glycogen, pectin, cellulose or polypeptide such as collagen, globulin), cysteine or a peptide with a high cysteine content or a phospholipid is used as a shell or shells. In the sense of the invention, the step of adding a shell to the core means completely surrounding the core, covering only some distinct areas and preferably all ranges between these situations.

Advantageous variants of current methods for the production of targeting contrast agents or targeting therapeutic agents are defined in the dependent claims.

In detail, the present invention provides several particularly advantageous variants as described below.

The “core”: material suitable as contrast-enhancing part and/or the therapeutic part of the present targeting contrast agent. Said core has a covalent and ionic bond with the ligand because of the particular structure of the polypeptides used as linking unit.

The “shell or shells”: material that can allow a good dispersion of the targeting contrast agent is able to decrease its toxicity or can prevent adverse effects, depending on the material that is used as a shell. If nanoparticles are used as the core, the use of an appropriate shell (e.g. a shell of ZnS) can reduce the number of surface defects of the nanoparticles. These defects considerably reduce the contrast generated by the nanoparticles. A reduction of the number of defects therefore leads to better targeting contrast-enhancing agents.

In the context of the present invention, “core” and “modified core” can be used as synonyms and a “modified core” is a core modified by at least one attached binding unit.

In the context of the present invention, “shell or shells” and “modified shell or shells” can be used as synonyms and “modified shells” are shells modified by at least one attached binding unit.

“Binding units”, in the context of the present invention, are understood to be at least one molecule of an aryl boronic acid, a hypervalent aryl siloxane or iodobenzene. In preferred embodiments of the present invention, a combination of shells, modified shells and modified cores are binding units (e.g. a modified core partially covered by a PEG shell, a core partially covered by a PEG shell and partially covered by a carboxylic acid modified shell linked to aryl boronic acid).

The expression “ligand” can be used as a synonym in the context of the present invention with a binder or preferably with a biologically active ligand.

In the context of the present invention, an “appropriate catalyst” is e.g. a Cu-based catalyst. Said catalyst allows the synthesis of targeting contrast agents by covalently linking a ligand bearing at least one histidine unit (e.g. poly-HIS tag) to an aryl boronic acid, a hypervalent aryl siloxane or an iodobenzene, attached to a core or to a shell or shells added to the core, preferably in mild conditions.

“Mild conditions” are understood to mean preferably art-known conditions under which the ligand will retain its activity and specificity, respectively, e.g. conditions in aqueous solutions or blood or serum-like solutions, physiological pH values and room temperature.

The present invention further relates to targeting contrast agents and targeting therapeutic agents and their use.

The targeting contrast agent has the following characteristics describing the invention by way of non-limiting example.

Depending on the contrast-enhancing material, the targeting contrast agent can be applied in different imaging procedures such as MRI, US, SPECT, CT, PET, optical imaging or multimodalit approaches like PET/CT.

The targeting contrast agent comprises a contrast-enhancing core (e.g. magnetic nanoparticles) or a therapeutic core that can be covered by one or more shells to improve stability and/or biocompatibility and/or to reduce toxicity in vivo (e.g. PEG shell).

If nanoparticles are used as the core, the size of these particles may vary from about 1 nm to 200 nm. In preferred embodiments of the present invention, the size of the particles may vary from 1 nm to 100 nm.

If polymers are used as shells, the molecular weight of these polymers may vary from 200 g/mol to 200,000 g/mol. In preferred embodiments of the present invention, the molecular weight of these polymers may vary from 200 g/mol to 100,000 gμmol.

The targeting contrast agent comprises a targeting ligand.

In further embodiments, the targeting contrast agents or the targeting therapeutic agents comprise a modified core or modified shell or shells linked to the ligand.

The targeting contrast agent comprises a ligand, which is able to specifically recognize a target molecule in vivo or in vitro.

One advantage of the present synthesis of targeting contrast agents, wherein the modified core or the modified shell or shells are covalently bonded to the ligand (binder) by a catalyzed reaction of boronic acids, hypervalent aryl siloxane or iodobenzene with histidine, is that the bond which is formed by this reaction is particularly stable, even in vivo. Thus, the ligand and the modified core remain linked in vivo, avoiding contrasting of undesired areas (e.g. tissues).

The described bond between the modified core or the modified shell or shells and the ligand can be generated under mild reaction conditions in aqueous media, which allows the ligand to keep its full biological activity. This is possible because the reaction can be catalyzed by a copper catalyst in water at room temperature and because the modified cores or the modified shell or shells and the obtained targeting contrast agents are water or blood or serum-soluble. These mild reaction conditions allow the ligands not to be denatured.

“Linking” of the modified core or the modified shell or shells and the ligand can be performed by using a polyhistidine tag (“HIS tag”: a stretch of 6 histidine amino acids) synthetically attached to the ligand. Biomolecules like peptides, proteins, enzymes and antibodies are often routinely synthesized with such a polyhistidine tag that helps purifying these biomolecules via e.g. affinity chromatography. The present invention allows use of these polyhistidine tags to link at least one ligand to the modified core or the modified shell or shells. Thus, there is no need to add another tag to the ligand. The synthesis of the ligands is therefore simplified. In addition, the polyhistidine tags attached to the ligands after synthesis do not have to be digested or split off during an additional purification step after synthesis of the ligands.

“Linking” of the modified core or the modified shell or shells to the ligand can be performed site-specifically, e.g. at the HIS tag site. Therefore, the recognition center of the ligand will retain its activity. Since the polyhistidine tags can be fixed everywhere on the ligand in a controlled and selective way (for example, selectively on any given amino acid of an amino acid sequence, serving as ligand), the ligand keeps its activity, thus avoiding the deactivation of the ligand and thus also avoiding the linking of the modified cores or the modified shell or shells to an undesired site in the ligand.

The described methods can be translated to targeting therapeutic agents as well. The methods described in this invention are potentially applicable to any ligand and any core, because of the mild reaction conditions, providing a very versatile and easily adaptable system for the preparation of any type of targeting contrast agent or targeting therapeutic agent.

A most preferred variant of the present targeting contrast agent is described schematically in FIG. 1.

### DESCRIPTION OF FIGURES IN DETAIL

FIG. 1:

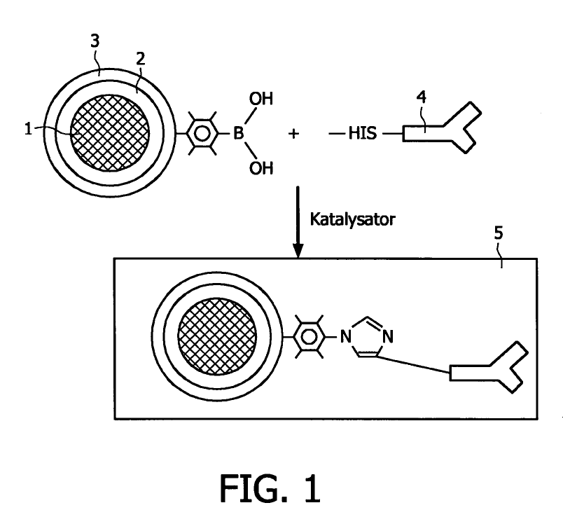

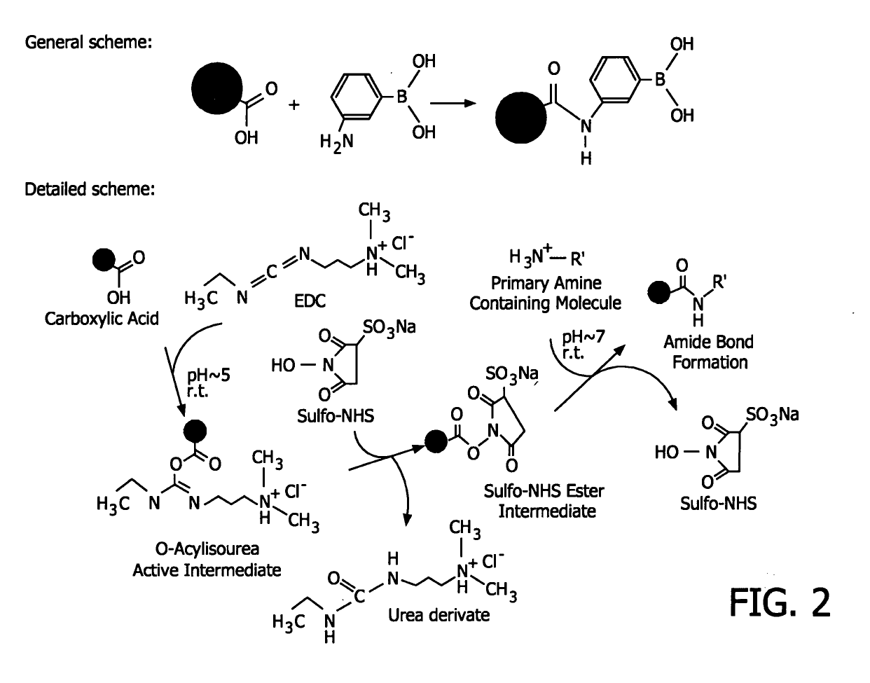

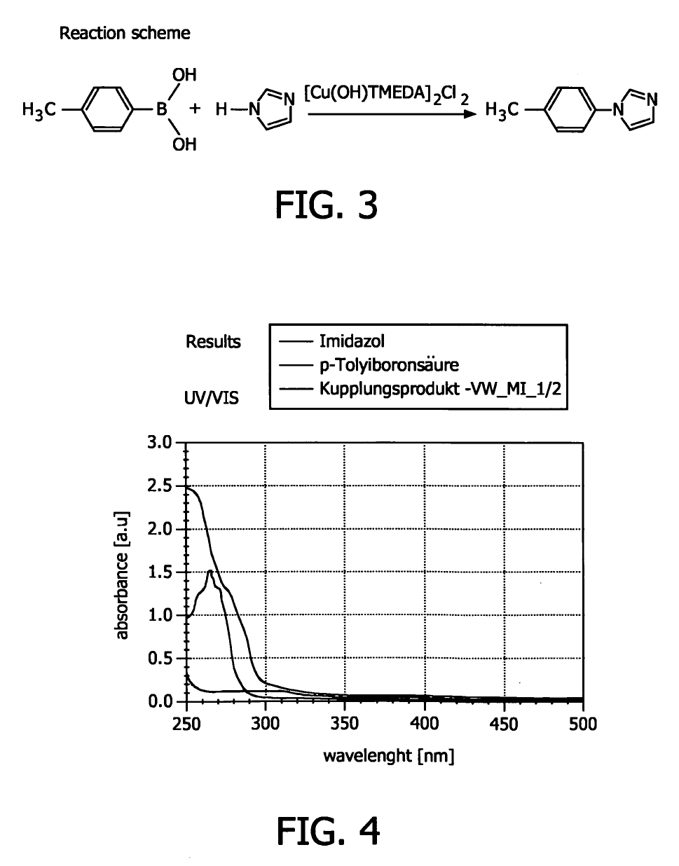

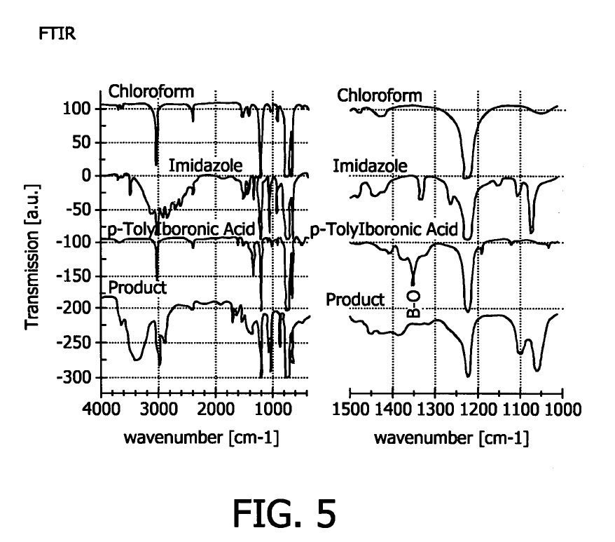

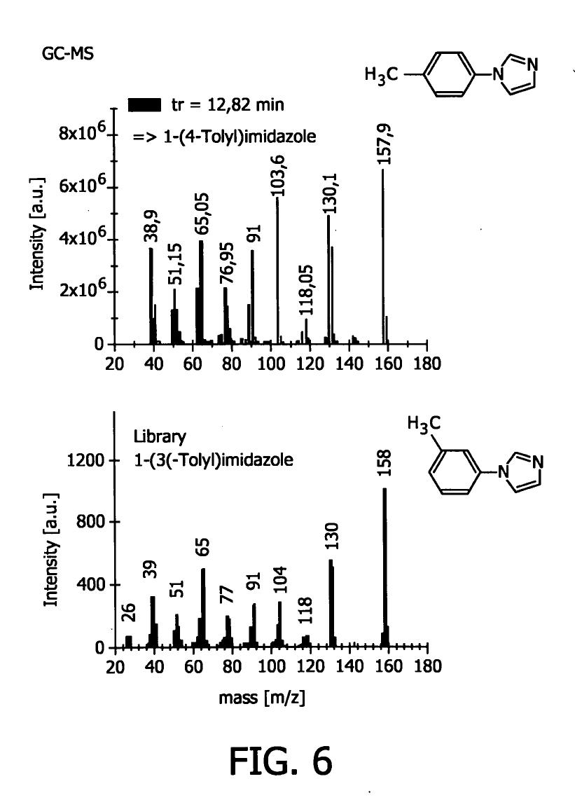

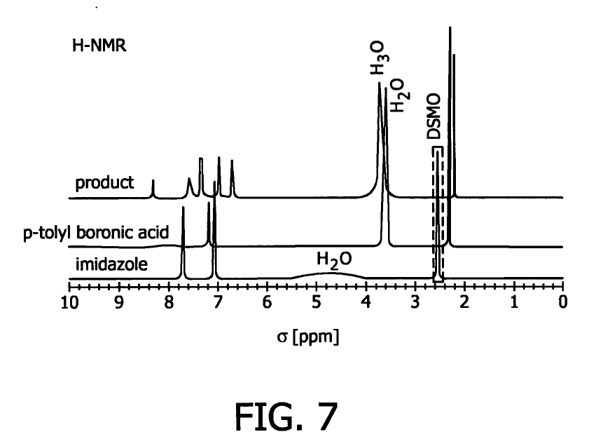

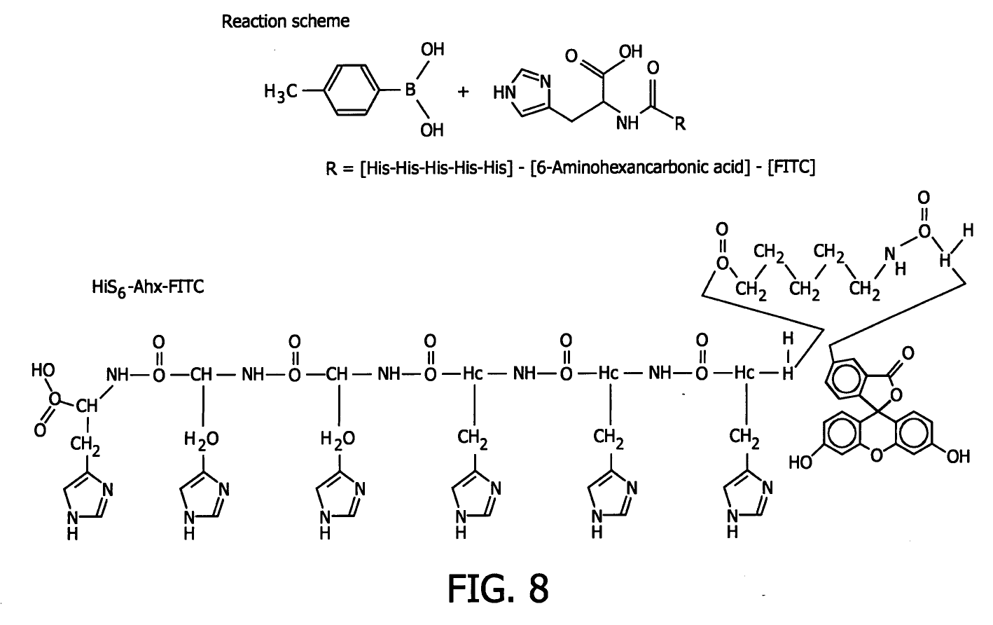

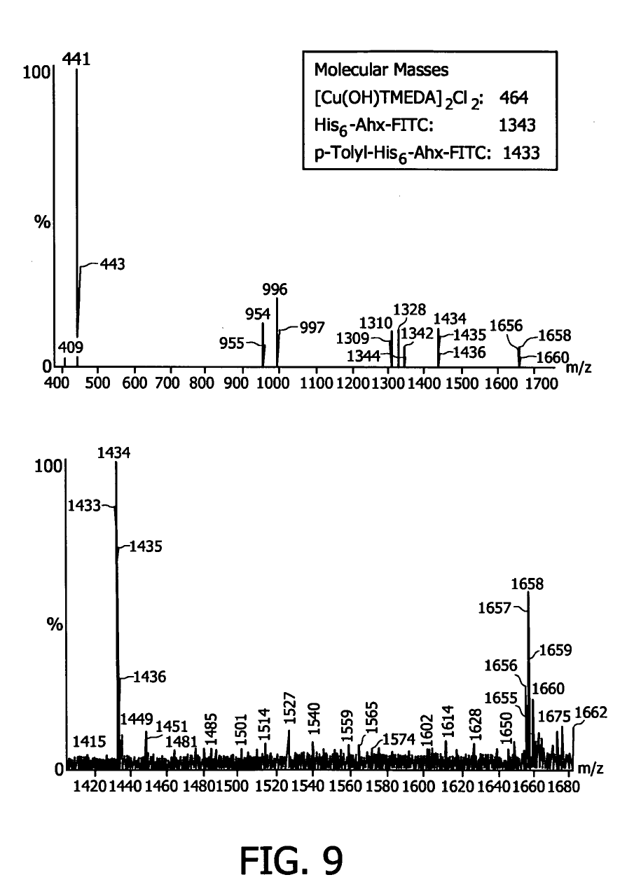

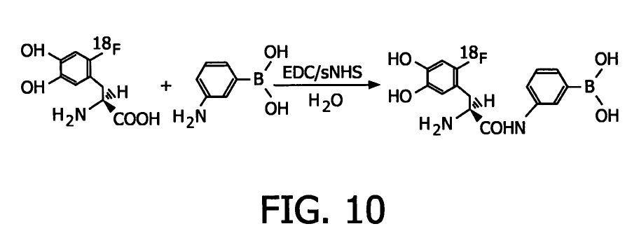

Core (1): e.g. (not limited to these) contrast-enhancing material; or therapeutical material for:

MRI: e.g. (not limited to these) ferro, antiferro, ferrimagnetic or superparamagnetic material such as iron (Fe), iron oxide γ-Fe2O3 or Fe₃O₄ or ferrite with spinel structure MFe₂O₄ (M=Mn, Co, Ni, Cu, Zn, Cd) or ferrite with garnet structure M₃Fe₅O₁₂ (M=Y, La, Ce, Pr, Nd, Sm, Eu, Gd, Th, Dy, Ho, Er, Tm, Yb, Lu) or ferrite with a magnetoplumbite structure MFe₁₂O₁₉ (M=Ca, Sr, Ba, Zn) or other hexagonal ferrite structures such as e.g. Ba₂M₂Fe₁₂O₂₂ (M=Mn, Fe, Co, Ni, Zn, Mg); in all cases, the core can be doped with additional 0.01 to 5.00 mol % of Mn, Co, Ni, Cu, Zn or F.

Paramagnetic ion (e.g. lanthanide, manganese, iron, copper)-based contrast-enhancing units, e.g. gadolinium chelates such as Gd(DTPA), Gd(BMA-DTPA), Gd(DOTA), Gd(DO3A); oligomeric structures; macromolecular structures such as albumin Gd(DTPA)20-35, dextran Gd(DTPA), Gd(DTPA)-24-cascade polymer, polylysine-Gd(DTPA), MPEG polylysine-Gd(DTPA); dendrimeric structures of lanthanide-based contrast-enhancing units; manganese-based contrast-enhancing units such as Mn(DPDP), Mn(EDTA-MEA), poly-Mn(EED-EEA), and polymeric structures; liposomes as carriers of paramagnetic ions, e.g. liposomal Gd(DTPA); non-proton imaging agents;

Optical: e.g. (not limited to these) luminescent materials such as nanophosphors (e.g. rare-earth doped YPO₄ or LaPO₄) or semiconducting nanocrystals (referred to as quantum dots; e.g. CdS, CdSe, ZnS/CdSe, ZnS/CdS); carbocyanine dyes; tetrapyrrole-based dyes (porphyrins, chlorins, phthalocyanines and related structures); delta aminolevulinic acid; fluorescent lanthanide chelates; fluorescein or 5-aminofluorescein or fluorescein isothiocyanate (FITC) or other fluorescein-related fluorophors such as Oregon Green, naphthofluorescein;

US: e.g. (not limited to these) shell (e.g. protein, lipid, surfactant or polymer) encapsulated gas (e.g. air, perfluoropropane, dodecafluorocarbon, sulphur hexafluoride, perfluorocarbon) bubbles (such as Optison from Amersham, Levovist from Schering); shell (e.g. protein, lipid, surfactant or polymer) encapsulated droplets; nanoparticles (e.g. platinum, gold, tantalum);

X-Ray: e.g. (not limited to these) iodinated contrast-enhancing units such as e.g. ionic and non-ionic derivatives of 2,4,6-tri-iodobenzene; barium sulfate-based contrast-enhancing units; metal ion chelates such as e.g. gadolinium-based compounds; boron clusters with a high proportion of iodine; polymers like iodinated polysaccharides, polymeric triiodobenzenes; particles from iodinated compounds displaying low water solubility; liposomes containing iodinated compounds; iodinated lipids such as triglycerides, fatty acids;

PET: e.g. (not limited to these) ¹¹C, ¹³N, ¹⁵O, ^{66/8}Ga, ⁶⁰Cu, ⁵²Fe, ⁵⁵Co, ^{61/2/4}Cu, ^{62/3}Zn, ^{70/1/4}AS, ^{75/6}Br, ⁸²Rb, ⁸⁶Y, ⁸⁹zr, ¹¹⁰In. ^{120/4}I, ¹²²Xe and ¹⁸F-based tracers such as e.g. ¹⁸F-FDG (glucose metabolism); ¹¹C-methionine, ¹¹C-tyrosine, ¹⁸F-FMT, ¹⁸F-FMT or ¹⁸F-FET (amino acids); ¹⁸F-FMISO, ⁶⁴Cu-ATSM (hypoxia); ¹⁸F-FLT, ¹¹C-thymidine, ¹⁸F-FMAU(proliferation);

SPECT: e.g. (not limited to these) contrast-enhancing units based on radionucleotides such as e.g. ^{99m}Tc, ^{123/131}I, ⁶⁷Cu, ¹¹¹In, ²⁰¹Tl;

Therapeutic material: e.g. (not limited to these) toxins, radioisotopes and chemotherapeutics; UV-C emitting nanoparticles such as e.g. YPO₄:Pr; photodynamic therapy (PDT) agents such as e.g. compounds based on expanded porphyrin structures; nucleotides for radiotherapy such as e.g. ¹⁵⁷Sm, ¹⁷⁷Lu, ^{212/3}Bi, ^{186/6}Re, ⁶⁷Cu, ⁹⁰Y, ¹³¹I, ^{114m}In, At, Ra, Ho;

Smart contrast-enhancing units such as e.g. (not limited to these) chemical exchange saturation transfer (CEST); thermosensitive MRI contrast agents (e.g. liposomal); pH-sensitive MRI contrast agents; oxygen pressure or enzyme-responsive MRI contrast agents; metal ion concentration-dependent MRI contrast agents;

Multi-modality: combinations of the above

Shell or shells (2): e.g. (not limited to these) may comprise carboxylic acids, acid halides, amines, acid anhydrides, activated esters, maleimides, isothiocyanates, gold, siO₂, a polyphosphate (e.g. calcium polyphosphate), an amino acid (e.g. cysteine), an organic polymer (e.g. polyethylene glycol/PEG, polyvinyl alcohol/PVA, polyamide, polyacrylate, polyurea), an organic functional polymer (e.g. 1,2-distearoyl-sn-glycero-3-phosphoethanolamine-N-[carboxy(polyethylene glycol)2000]ammonium salt), a biopolymer (e.g. polysaccharide such as dextran, xylan, glycogen, pectin, cellulose or polypeptide such as collagen, globulin), cysteine or a peptide with a high cysteine content or a phospholipid.

One to several shells, preferably 1 to 100 shells (2) can be added to the core, more preferably 1 to 50 inner shells. Each of these shells (which may comprise a monolayer or a polylayer of an appropriate material in preferred embodiments of the present invention) has a thickness of about 0.5 nm to 100 nm. In a preferred embodiment of the present invention, each shell has a thickness of about 0.5 nm to 500 nm and can be made of different materials or of the same material. Furthermore, the shell can cover the core at least partially.

Binding unit or units (3): e.g. (not limited to these) aryl boronic acids, a shell comprising aryl boronic acids functionality that mediates a covalent coupling with a histidine unit (e.g. poly-HIS tag) of a bioligand (e.g. antibody or antibody fragment, peptide, small molecule)

e.g. (not limited to these) hypervalent aryl siloxanes, a shell comprising hypervalent aryl siloxanes that mediates a covalent coupling with a histidine unit (e.g. poly-HIS tag) of a bioligand (e.g. antibody or antibody fragment, peptide, small molecule)

e.g. (not limited to these) iodobenzene, a shell comprising iodobenzenes or at least one iodobenzene bond to a shell that mediates a covalent coupling with a histidine unit (e.g. poly-HIS tag) of a bioligand (e.g. antibody or antibody fragment, peptide, small molecule)

In addition, further biomolecules such as proteins can be incorporated, enabling the passage of the complete assembly through e.g. cell membranes (e.g. the HIV tag peptide, etc.), increasing the biocompatibility or decreasing the toxicity.

Ligand (4):

e.g. (not limited to these) a ligand, which induces, through its specific recognition mechanism, the enrichment of contrast agent in distinct tissue or target regions of interest (e.g. by antibody antigen interaction)

e.g. (not limited to these) a ligand, which has attached a poly-HIS tag

Targeting units may be:

e.g. (not limited to these) antibodies (monoclonal, polyclonal, mouse, mouse-human chimeric, human, single-chain, diabodies, etc.) such as Trastuzumab (breast cancer), Rituximab (non-Hodgkin lymphoma), Alemtuzumab (chronical lymphozytic leukemia); Gemtuzumab (acute myelogene leukemia); Edrecolomab (colon cancer); Ibritumomab (non-Hodgkin lymphoma); Cetuximab (colon cancer); Tositumomab (non-Hodgkin lymphoma); Epratuzumab (non-Hodgkin lymphoma); Bevacizumab (lung and colon cancer); anti-CD33 (acute myelogene leukemia); Pemtumomab (ovarian and stomach cancer); Mittumomab (lung and skin cancer); anti-MUC 1 (adenocarcinoma); anti-CEA (adenocarcinoma); anti-CD 64 (plaques), etc.

e.g. (not limited to these) peptides, polypeptides, peptidomimetics, such as somatostatin analogs, vasoactive peptide analogs, neuropeptide Y, RGD peptides, etc.

e.g. (not limited to these) proteins such as annexin V, tissue plasminogen activator protein, transporter proteins, etc.

e.g. (not limited to these) macromolecules, e.g. hyaluronan, apcitide, dermatan sulfate

e.g. (not limited to these) nucleic acids such as apatamers, anti-sense DNA/RNA,/PNA, small interfering RNAs, etc.

e.g. (not limited to these) lipids such as phospholipids, etc.

e.g. (not limited to these) lectins, e.g. leukocyte stimulatiry lectin

e.g. (not limited to these) saccharides

Catalysts

e.g. (not limited to these) catalyzes the reaction of an aryl boronic acid functionality with an imidazole functionality and allows a reaction window in that the bioligand is not damaged during the coupling reaction (e.g. aqueous solution, pH=7, room temperature), e.g. [Cu(OH)TMEDA]₂Cl₂; (TMEDA-tetramethyl ethylene diamine) see, for example, Collman, Zhong, Zeng, Costanza, J. Org. Chem., 2001, 66, 1528-1531

e.g. (not limited to these) catalyzes the reaction of a hypervalent aryl siloxane with an imidazole functionality and allows a reaction window in that the bioligand is not damaged during the coupling reaction, e.g. Cu(AcO)₂, see, for example, Lam, Deudon, Averill, Li, He, DeShong, Clark, J. Am. Chem. Soc., 2000, 122, 7600-7601

e.g. (not limited to these) catalyzes the reaction of an iodobenzene with an imidazole functionality, e.g. [Cu(OH)TMEDA]₂Cl₂; see, for example, Lam, Deudon, Averill, Li He, DeShong, Clark, J. Am. Chem. Soc., 2000, 122, 7600-7601

targeting contrast agent or therapeutic agent (5)

e.g. (not limited to these) consists of contrast-enhancing or therapeutic core, shells with different functionality, a coupling unit (phenyl imidazole) and a specific targeting ligand

FIG. 2:

Reaction scheme for the surface modification of a contrast-enhancing unit (COOH coated CdSe/ZnS quantum dots) with phenyl boronic acid, by a one-pot reaction of carboxylic acids, linked to the core, with 1-ethyl-3-(dimethyl aminopropyl) carbodiide hydrochloride (EDC) to form a o-acylisourea intermediate (room temperature, pH≈5). This intermediate reacts with sulfo-NHS to give a sulfo-NHS ester intermediate. The excess of EDC is quenched by the addition of 2-mercaptoethanol. Finally, the reaction with 3-amino phenyl boronic acid leads to the desired amide bond (r.t, pH≈7).

FIG. 3:

Reaction scheme for the coupling of p-tolyl boronic acid with imidazole performed according to Collmann et al. (J. Org. Chem., 2001, 66, 1528-1531).

FIG. 4:

The absorbance of imidazole, p-tolyl boronic acid and the coupling product is measured (in arbitrary units) as a function of the wavelength (in nm) of the incident radiation between 250 and 500 nanometers. The differences seen between the UV/Vis spectra of the two starting products (imidazole and p-tolyl boronic acid) and the spectrum of the coupling product, obtained after reaction, prove that the coupling occurs under the described conditions.

FIG. 5:

The transmission of chloroform, imidazole, p-tolyl boronic acid and the coupling product is measured (in arbitrary units) as a function of the wavenumber (in cm⁻¹) of the incident radiation between 0 cm⁻¹ and 4000 cm⁻¹, and between 1000 cm⁻¹ and 1500 cm⁻¹. The differences seen between the FTIR spectra of the solvent (chloroform), the two starting products (imidazole and p-tolyl boronic acid) and the spectrum of the coupling product, obtained after reaction, prove that the coupling occurs under the described conditions.

FIG. 6:

The intensity of the signals recorded for imidazole, p-tolyl boronic acid and the coupling product is measured (in arbitrary units) as a function of the mass (in m/z units) by mass spectroscopy after the isolation of 1-(4-tolyl) imidazole (obtained by the coupling reaction) by gas chromatography. The similarity between the GC/MS spectrum of 1-(4-tolyl) imidazole, obtained by the coupling of p-tolyl boronic acid with imidazole under the described conditions, and the GC/MS spectrum of 1-(3-tolyl) imidazole, found in a spectrum library, proves that the desired coupling product is obtained.

FIG. 7:

The intensity of the signals of imidazole, p-tolyl boronic acid and the coupling product is measured as a function of the chemical shift (in ppm) by NMR. The differences seen between the NMR spectra of the solvent (chloroform), the two starting products (imidazole and p-tolyl boronic acid) and the spectrum of the coupling product, obtained after reaction, prove that the coupling occurs under the described conditions.

FIG. 8:

Reaction scheme for the coupling of p-tolyl boronic acid with (His)₆-Ahx-FITC, via the reaction of p-tolyl boronic acid with a histidine unit of the (His)₆-Ahx-FITC tag, catalyzed overnight by Cu(OH)TMEDA]₂Cl₂ at room temperature.

FIG. 9:

The intensity of the signals recorded for the product obtained after reaction is measured (in arbitrary units) as a function of the mass (in m/z units) by MALDI-TOF (matrix-assisted laser desorption ionization—time of flight) mass spectroscopy. The MALDI-TOF spectrum of the product, obtained after the coupling reaction of p-tolyl boronic acid with (His)₆-Ahx-FITC, proves that the coupling occurs under the described conditions. The peak at m/z=1433 corresponding to the desired coupling product (p-Tolyl-His₆-Ahx-FTC) proves the formation of this product.

FIG. 10:

Reaction scheme for modification of a core (¹⁸F-marked molecule) with phenyl boronic acid, by a one-pot reaction of carboxylic acids, linked to the contrast-enhancing unit, with 1-ethyl-3-(dimethyl aminopropyl) carbodiide hydrochloride to form a o-acylisourea intermediate (r.t., pH≈5). This intermediate reacts with sulfo-NHS to give a sulfo-NHS ester intermediate. The excess of EDC is quenched by the addition of 2-mercaptoethanol. Finally, the reaction with 3-amino phenyl boronic acid leads to the desired amide bond (r.t., pH≈7).

### EXAMPLES

### Example 1

CdSe/ZnS quantum dots (cores) were surface-modified with a carboxylic acid functionality by an acid by means of a water-soluble polymer bearing a carboxylic acid function at one end and a 1,2-distearoyl-sn-glycero-3-phosphoethanolamine function at the other end.

The COOH-coated quantum dots were obtained by mixing (4 h at 50° C.):
100 μl CdSe/ZnS (in chloroform, 1 w/v %)
100 μl chloroform
200 μl DPPC (5 mM)−DPPC=1,2-dipalmitoyl-sn-glycero-3-phosphocholine
200 μl DSPE-PEG2000-COOH (5 mM)-DSPE-PEG2000-COOH: 1,2-distearoyl-sn-glycero-3-phosphoethanolamine-N-[Carboxy(polyethylene glycol)2000]ammonium salt,

and finally removing the chloroform by vacuum and dispersing the COOH-coated quantum dots in water by ultrasonic treatment.

The 1,2-distearoyl-sn-glycero-3-phosphoethanolamine-N-[carboxy(polyethylene glycol)2000]ammonium salt binds to the surface of the nanoparticles by hydrophobic interactions (or adsorption) by the 1,2-distearoyl-sn-glycero-3-phosphoethanolamine end group. Furthermore, the 1,2-distearoyl-sn-glycero-3-phosphoethanolamine-N-[carboxy(polyethylene glycol)2000]ammonium salt provides a carboxy function, which is protonated, at an acid pH, to obtain a carboxylic acid.

DPPC is used as a dummy (or spacer) to leave spaces between the COOH functions fixed on the nanoparticles. Actually, the covering of the whole nanoparticle surface only by COOH functions could have adverse effects by creating interactions, and therefore contrast, in undesired tissues or undesired areas of the body.

1) Surface Modification of the Shell with Phenyl Boronic Acid

The contrast-enhancing unit can be surface-modified with boronic acid functionality by coupling via a carboxylic acid.

Other examples would be e.g. coupling via an activated ester, via maleimide or via isothiocyanate.

This is experimentally done by modifying water-soluble CdSe/ZnS quantum dots:
55 μl water
40 μl 10× PBS solution (PBS=phosphate buffer saline: 0.01 M phosphate buffer, 0.0027 M potassium chloride, 0.137 M sodium chloride, pH 7.4)
100 μl 0.1 M EDC solution (EDC=1-ethyl-3-dimethyl aminopropyl) carbodiimide hydrochloride)
5 μl 20 mM sulfo-NHS solution (N-hydroxy sulfosuccinimide sodium salt)
200 μl 2 μM CdSe/ZnS (COOH terminated) solution
Incubation at r.t. (30 min)
10 μl 2-mercaptoethanol
mixing for 15 min
50 μl 20 mM 3-amino phenyl boronic acid solution
mixing at r.t. (2 h)
separation of QDs by centrifugation

Reaction scheme, see FIG. 2.

1) Coupling of p-Tolyl Boronic Acid with Imidazole

As an initial step, the reaction of tolyl boronic acid with imidazole described in literature was successfully reproduced. Synthesis performed according to Collmann et al., (J. Org. Chem., 2001, 66, 1528-1531).

Reaction scheme, see FIG. 3.

2) Coupling of p-tolyl Boronic Acid with (His)₆-Ahx-FITC

The catalyzed reaction of phenyl boronic acids with imidazole can be adopted for the reaction of phenyl boronic acids with peptides with a poly-HIS tag, which could be proved experimentally:

### Synthesis:

19 μl 100 μM [Cu(OH)TMEDA]₂Cl₂ solution

38 μl 1 mM p-tolyl boronic acid solution

31.9 μl His6-Ahx-FITC (0.8 mg/ml) (His6=oligohistidine; Ahx=6-amino hexacarbonic acid; FITC=fluorescein isothiocyanate (IsomerIK)

1911.1 μl water

incubation in oxygen atmosphere (overnight at r.t.)

Reaction scheme, see FIG. 4.

### Example 2

### ¹⁸F-Marked Molecule Modified by Boronic Acid

This is experimentally performed by:
55 μl water
40 μl 10× PBS solution (PBS=phosphate buffer saline: 0.01 M phosphate buffer, 0.0027 M potassium chloride, 0.137 M sodium chloride, pH 7.4)
100 μl 0.1 M EDC solution (EDC=1-ethyl-3-(dimethyl aminopropyl) carbodiimide hydrochloride)
5 μl 20 mM sulfo-NHS solution (N-hydroxy sulfosuccinimide sodium salt)
200 μl 2 μM F-18-L-DOPA solution
Incubation at r.t. (30 min)
10 μl 2-mercaptoethanol
mixing for 15 min
50 μl 20 mM m-amino phenyl boronic acid
mixing at r.t. (2 h)
removal of by-products by centrifugation

1. A method for the production of a targeting contrast agent or a therapeutic agent, the method comprising the steps of:
a) providing a core;
b) optionally adding a shell to the core;
c) modifying the core or the shell by attaching at least one molecule of a binding unit; and
d) linking at least one ligand, bearing at least one imidazole functionality, to the modified core or the modified shell by using an appropriate catalyst.

2. The method according to claim 1, wherein, in step b), more than one shell is added to the core.

3. The method according to claim 1, wherein the shell or shells comprise a monolayer or a polylayer.

4. The method according to claim 1, wherein each shell comprises the same material or a different material.

5. The method according to claim 1, wherein the shell or shells cover the core at least partially.

6. A method according to claim 1, wherein the material used as the core is selected from:
ferro, antiferro, ferrimagnetic or superparamagnetic materials such as iron (Fe), iron oxide γ-Fe2O3 or Fe₃O₄ or ferrite with spinel structure MFe₂O₄ (M=Mn, Co, Ni, Cu, Zn, Cd) or ferrite with garnet structure M₃Fe₅O₁₂ (M=Y, La, Ce, Pr, Nd, Sm, Eu, Gd, Tb, Dy, Ho, Er, Tm, Yb, Lu), or ferrite with a magnetoplumbite structure MFe₁₂O₁₉ (M=Ca, Sr, Ba, Zn), or other hexagonal ferrite structures such as Ba₂M₂Fe₁₂O₂₂ (M=Mn, Fe, Co, Ni, Zn, Mg); wherein, in all cases, the core can be doped with additional 0.01 to 5.00 mol % of Mn, Co, Ni, Cu, Znor F; paramagnetic ion (e.g. lanthanide, manganese, iron, copper)-based contrast-enhancing units e.g. gadolinium chelates such as Gd(DTPA), Gd(BMA-DTPA), Gd(DOTA), Gd(DO3A); oligomeric structures; macromolecular structures such as albumin Gd(DTPA)20-35, dextran Gd(DTPA), Gd(DTPA)-24-cascade polymer, polylysine-Gd(DTPA), MPEG polylysine-Gd(DTPA); dendrimeric structures of lanthanide-based contrast-enhancing units; manganese-based contrast-enhancing units such as Mn(DPDP), Mn(EDTA-MEA), poly-Mn(EED-EEA), and polymeric structures; liposomes as carriers of paramagnetic ions such as liposomal Gd(DTPA); non-proton imaging agents.

7. A method according to claim 1, wherein the material used as the core is selected from:
luminescent material such as nanophosphors (e.g. rare-earth doped YPO₄ or LaPO₄) or semiconducting nanocrystals (referred to as quantum dots; e.g. CdS, CdSe, ZnS/CdSe, ZnS/CdS); carbocyanine dyes; tetrapyrrole-based dyes (porphyrins, chlorins, phthalocyanines and related structures); delta aminolevulinic acid; fluorescent lanthanide chelates; fluorescein or 5-aminofluorescein or fluorescein-isothiocyanate (FITC) or other fluorescein-related fluorophors such as Oregon Green, naphthofluorescein.

8. A method according to claim 1, wherein the material used as the core is selected from:
encapsulated gas (e.g. air, perfluoropropane, dodecafluorocarbon, sulphur hexafluoride, perfluorocarbon) bubbles (such as Optison from Amersham, Levovist from Schering); encapsulated droplets; nanoparticles (e.g. platinum, gold, tantalum).

9. A method according to claim 1, wherein the material used as the core is selected from:
iodinated contrast-enhancing units such as ionic and non-ionic derivatives of 2,4,6-tri-iodobenzene; barium sulfate-based contrast-enhancing units; metal ion chelates such as gadolinium-based compounds; boron clusters with a high proportion of iodine; polymers such as iodinated polysaccharides, polymeric triiodobenzenes; particles from iodinated compounds displaying a low water solubility; liposomes containing iodinated compounds; iodinated lipids like triglycerides, fatty acids.

10. A method according to claim 1, wherein the material used as the core is selected from:
¹¹C, ¹³N, ¹⁵O, ^{66/8}Ga, ⁶⁰Cu, ⁵²Fe, ⁵⁵Co, ^{61/2/4}Cu, ^{70/1/4}As, ^{75/6}Br, ⁸²Rb, ⁸⁶Y, ⁸⁹Zr, ¹¹⁰In, ^{120/4}I, ¹²²Xe and ¹⁸F-based tracers such as ¹⁸F-FDG (glucose metabolism); ¹¹C-methionine, ¹¹C-tyrosine, ¹⁸F-FMT, ¹⁸F-FMT or ¹⁸F-FET (amino acids); ¹⁸F-FMISO, ⁶⁴Cu-ATSM (hypoxia); ¹⁸F-FLT, ¹¹C-thymidine, ¹⁸F-FMAU (proliferation).

11. A method according to claim 1, wherein the material used as the core is selected from:
contrast-enhancing units based on radionucleotides such as ^{99m}Tc, ^{123/5/131}I, ⁶⁷Cu, ⁶⁷Ga, ¹¹¹In, ²⁰¹Tl.

12. A method according to claim 1, wherein the material used as the core is selected from:
toxins, radioisotopes and chemotherapeutics; UV-C emitting nanoparticles such as YPO₄:Pr; photodynamic therapy (PDT) agents such as compounds based on expanded porphyrin structures; nucleotides for radiotherapy such as ¹⁵⁷Sm, ¹⁷⁷Lu, ^{212/3}Bi, ^{186/8}Re, ⁶⁷Cu, ⁹⁰Y, ¹³¹I, ^{114m}In, At, Ra, Ho.

13. A method according to claim 1, wherein the material used as the core is selected from:
chemical exchange saturation transfer (CEST); thermosensitive MRI contrast agents (e.g. liposomal); pH-sensitive MRI contrast agents; oxygen pressure or enzyme-responsive MRI contrast agents; metal ion concentration-dependent MRI contrast agents.

14. A method according to claim 1, wherein the material used as the core is a combination of two or more materials.

15. A method according to claim 1, wherein the material used as a shell or shells is selected from:
carboxylic acids, acid halides, amines, acid anhydrides, activated esters, maleimides, isothiocyanates, gold, SiO₂, lipids, surfactants, a polyphosphate (e.g. calcium polyphosphate), an amino acid (e.g. cysteine), an organic polymer (e.g. polyethylene glycol/PEG, polyvinyl alcohol/PVA, polyamide, polyacrylate, polyurea), an organic polymer with functional end groups (e.g. 1,2-distearoyl-sn-glycero-3-phosphoethanolamine-N-[carboxy(polyethylene glycol)2000]ammonium salt), a biopolymer (e.g. polysaccharide such as dextran, xylan, glycogen, pectin, cellulose or polypeptide such as collagen, globulin), cysteine or a peptide with a high cysteine content or a phospholipid.

16. A method according to claim 1, wherein further components can be incorporated into the shell or shells.

17. A method according to claim 1, wherein the binding unit is an aryl boronic acid, a shell comprising aryl boronic acid functionality or at least one aryl boronic acid bond to a shell that couples covalently with a histidine unit of a ligand.

18. A method according to claim 1, wherein the binding unit is a hypervalent aryl siloxane, a shell comprising hypervalent aryl siloxane acid functionality or at least one hypervalent aryl siloxane bond to a shell that couples covalently with a histidine unit of a ligand.

19. A method according to claim 1, wherein the binding unit is an iodobenzene, a shell comprising iodobenzene functionality or at least one iodobenzene bond to a shell that couples covalently with a histidine unit of a ligand.

20. A method according to claim 1, wherein the core or the shell or shells and at least one ligand are linked by a covalent coupling between an aryl boronic acid and a histidine unit.

21. A method according to claim 1, wherein the core or the shell or shells and at least one ligand are linked by a covalent coupling between a hypervalent aryl siloxane and a histidine unit.

22. A method according to claim 1, wherein the core or the shell or shells and at least one ligand are linked by a covalent coupling between an iodobenzene and a histidine unit.

23. A method according to claim 1, wherein the material used as a ligand is selected from:
antibodies (monoclonal, polyclonal, mouse, mouse-human chimeric, human, single-chain, diabodies, etc.) such as Trastuzumab (breast cancer), Rituximab (non-Hodgkin lymphoma), Alemtuzumab (chronial lymphozytic leukemia); Gemtuzumab (acute myelogene leukemia); Edrecolomab (colon cancer); Ibritumomab (non-Hodgkin lymphoma); Cetuximab (colon cancer); Tositumomab (non-Hodgkin lymphoma); Epratuzumab (non-Hodgkin lymphoma); Bevacizumab (lung and colon cancer); anti-CD33 (acute myelogene leukemia); Pemtumomab (ovarian and stomach cancer); Mittumomab (lung and skin cancer); anti-MUC 1 (adenocarcinoma); anti-CEA (adenocarcinoma); anti-CD 64 (plaques; peptides, polypeptides, peptidomimetics such as somatostatin analogs, vasoactive peptide analogs, neuropeptide Y, RGD peptides; proteins such as Annexin V, tissue plasminogen activator proteins, transporter proteins; macromolecules such as hyaluronan, apcitide, dermatan sulphate; nucleic acids such as apatamers, anti-sense DNA/RNA,/PNA, small interfering RNAs; lipids such as phospholipids; lectins such as leukocyte stimulatory lectin and saccharides.

24. A method according to claim 1, wherein the catalyst used to link at least one ligand to the modified core or the modified shell is Cu(OH)TMEDA]₂Cl₂ or Cu(AcO)₂.

25. A method according to claim 1, wherein the catalyst used to catalyze the reaction of an aryl boronic acid functionality or an iodobenzene functionality with an imidazole functionality is Cu(OH)TMEDA]₂Cl₂.

26. A method according to claim 1, wherein the catalyst used to catalyze the reaction of a hypervalent aryl siloxane with an imidazole functionality is Cu(AcO)₂.

27. Targeting contrast agents comprising a core, at least one shell and at least one ligand.

28. Targeting contrast agents or targeting therapeutic agents produced by means of a method according to claim 1.

29. Targeting contrast agents or targeting therapeutic agents according to claim 27 for use in diagnosis or therapy.

30. Targeting contrast agents or targeting therapeutic agents according to claim 27 for use in targeting molecular imaging.

31. Targeting contrast agents according to claim 27 for use in CT, MRI, PET, SPECT or US.

32. Use of the targeting contrast agents or targeting therapeutic agents according to claim 27 for the production of compounds suitable in diagnosis or therapy.

33. Use of the targeting contrast agents or targeting therapeutic agents according to claim 27 for the production of compounds suitable for targeting molecular imaging.

34. Use of the targeting contrast agents according to claim 27 for the production of compounds suitable in CT, MRI, PET, SPECT or US.
